(19) United States
(12) Patent Application Publication
(10) Pub. No.: US 2025/0218597 A1
(43) Pub. Date: Jul. 3, 2025

(54) METHOD FOR PROVIDING RECOVERY ESTIMATION

(71) Applicant: Summa Finland Oy, Oulu (FI)

(72) Inventors: Tommi Kinnunen, Helsinki (FI); Aleksi Juurikka, Oulu (FI); Wojciech Kurek, Wroclaw (PL); Juuso Nissilä, Oulu (FI); Marko Mäkinen, Oulu (FI)

(73) Assignee: Summa Finland Oy, Oulu (FI)

(21) Appl. No.: 18/966,773

(22) Filed: Dec. 3, 2024

(30) Foreign Application Priority Data
Dec. 27, 2023 (FI) 20236440

(51) Int. Cl. G16H 50/30; G16H 10/20; G16H 10/60

(52) U.S. Cl. CPC G16H 50/30; G16H 10/20; G16H 10/60

(57) ABSTRACT

A method for providing a recovery estimation value for a user related to an exposure to a load includes: providing a first set of questions to the user, via a computing arrangement; receiving, after the exposure to the load, a first set of respective answers from the user, to the first set of questions, at the computing arrangement; using the first set of questions and the respective answers as a first input to a first neural network, via the computing arrangement, wherein the first neural network is trained to provide a load estimation related to the physical exercise using the first input; and calculating from the load estimation the recovery estimation value, using the computing arrangement.

### TECHNICAL FIELD

The present disclosure relates to methods for providing a recovery estimation value for a user related to an exposure to a load. The present disclosure also relates to systems for providing a recovery estimation value for a user. The present disclosure further relates to methods for training a neural network model for providing a recovery estimation value for a user.

### BACKGROUND

Generally, across a wide range of sports and physical activities, such as running, walking, high jump, long jump, skiing, weightlifting, and the like, there exists a critical need to provide users with reliable information regarding the recovery time after exposure to a load, be it physical, mental, work-related, or due to an illness. Consider, for instance, the realm of sports training, where devices like heart rate monitors are commonly used to gauge the physical load of an exercise. While these devices contribute to physical load-based recovery estimations, there remains a challenge in achieving precision. To address this, one could measure hormonal values before, during, and/or after physical exercise, incorporating them into the recovery estimation process. However, the existing method encounters complications, notably the requirement for dedicated measurement devices for both physical load and hormonal values.

Therefore, in light of the foregoing discussion, there exists a need to overcome the aforementioned drawbacks.

### SUMMARY

The aim of the present disclosure is to provide a method for providing a recovery estimation value for a user related to an exposure to a load. The aim of the disclosure is achieved by a method which employs a series of steps designed to capture, analyze, and interpret user input in the context of load exposure. By presenting a set of targeted questions to the user and subsequently leveraging the responses as input for a trained neural network, the method achieves a nuanced load estimation (“modelled load”). The calculated load estimation is then judiciously utilized to derive a recovery estimation value, thereby fulfilling the primary aim of the disclosure. Advantageous features are set out in the appended dependent claims.

The aim of the present disclosure is to provide a system for providing a recovery estimation value for a user. The aim of the disclosure is achieved by a system in which a computing arrangement incorporates three neural networks, each trained to analyze various aspects of user input, including responses to questions, load measurements, and hormonal responses. The user device facilitates seamless interaction, collecting information from the user and relaying it to the computing arrangement. The collaborative functioning of these neural networks ensures a comprehensive and individualized approach to recovery estimation, fulfilling the overarching aim of the system. Advantageous features are set out in the appended dependent claims.

The aim of the present disclosure is to provide a method for training a neural network model for providing a recovery estimation value for a user. The aim of the disclosure is achieved by a method which encompasses both generic training, utilizing data from a plurality of users to create generally trained neural networks, and user-dependent training to customize these networks for individual users. This method ensures the neural network model is adept at capturing complex relationships between user input, load estimations, hormonal responses, and recovery estimations, thereby fulfilling the aim of the disclosure. Advantageous features are set out in the appended dependent claims.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 illustrates a flowchart depicting steps of a method for providing a recovery estimation value for a user related to an exposure to a load, in accordance with an embodiment of the present disclosure;

FIG. 2 illustrates a block diagram of a system for providing a recovery estimation value for a user, in accordance with an embodiment of the present disclosure;

FIG. 3 illustrates an exemplary flow diagram depicting an exemplary process flow of a system for providing a recovery estimation value for a user related to an exposure to a load, in accordance with an embodiment of the present disclosure; and

FIG. 4 illustrates a flowchart depicting steps of a method for training a neural network model for providing a recovery estimation value for a user related to an exposure to a load, in accordance with an embodiment of the present disclosure.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

In a first aspect, an embodiment of the present disclosure provides a method for providing a recovery estimation value for a user related to an exposure to a load, the method comprising:
providing a first set of questions to the user, via a computing arrangement;
receiving, after the exposure to the load, a first set of respective answers from the user, to the first set of questions, at the computing arrangement;
using the first set of questions and the respective answers as a first input to a first neural network, via the computing arrangement, wherein the first neural network is trained to provide a load estimation (“modelled load”) related to the physical exercise using the first input; and
calculating from the load estimation the recovery estimation value, using the computing arrangement.

In a second aspect, an embodiment of the present disclosure provides a system for providing a recovery estimation value for a user, the system comprising:
a computing arrangement comprising:
   a first neural network, which the first neural network is trained using as training data a first set of question answer pairs, load measurement data sets from a load measurement sensor and calculated load estimations related to the first set of question answer pairs,
   a second neural network, which the second neural network is trained using load estimations and related hormonal response values, and
   a third neural network, which the third neural network is trained using as training data a second set of question answer pairs, hormonal response values and recovery estimation values; and
a user device for providing the first and the second set of questions to the user, collecting answers from the user, providing the collected answers to the computing arrangement, receiving from the computing arrangement recovery estimation value and providing the recovery estimation value for the user.

In a third aspect, an embodiment of the present disclosure provides a neural network model for providing a recovery estimation value for a user and for use in the method of the first aspect, comprising:
a first neural network, wherein the first neural network is trained to provide a load estimation (“modelled load”) related to the physical exercise using a first set of questions and related answers as an input, via a computing arrangement;
a second neural network, wherein the second neural network is trained to provide a modelled hormonal response (“modelled hormones”) using the load estimation as an input, via the computing arrangement; and
a third neural network, wherein the third neural network is trained to provide a modelled recovery estimation (modelled recovery estimation is estimation value based on a model i.e “modelled recovery”) related to a second set of questions and related answers, via the computing arrangement, wherein the modelled recovery estimation is used as the recovery estimation value to be provided.

In a fourth aspect, an embodiment of the present disclosure provides a method for training a neural network model of the third aspect for providing a recovery estimation value for a user, wherein the method comprises:
receiving a first input training dataset comprising a set of measured loads and related first sets of questions and answers and using said first input training dataset to train the first neural network, via the computing arrangement;
receiving a second input training dataset comprising a set of measured loads and set of related hormonal responses and using said second input training dataset to train the second neural network, via the computing arrangement; and
receiving a third input training dataset comprising a set of measured hormones and a second sets of question and answers and using said third input training dataset to train the third neural network, via the computing arrangement;
   wherein,
the training of the neural network model comprises generic training using data from a plurality of users to create generally trained neural networks of the neural network model and user dependent training to customize the generally trained neural networks to be user dependent.

In a fifth aspect, an embodiment of the present disclosure provides use of the method of the first aspect for at least one of: providing recovery estimation after physical exercise, providing hormonal value estimations after a real or modelled load, providing possibility to make a forecast based on user's training plan constituted of the loading to take place in the future, a possibility to interpolate hormonal values and recovery estimation for any given past period of time where loading is measured with a sensor or documented by a set of question. Also, it is possible to detect a change in user's bodily homeostasis by comparing trained neural network's result to a real set of measured hormones and detect a discrepancy, which is essentially a sign of a major change in user's physiology, such as illness, change in performance level, in hormonal secretion etc. or a major change in lifestyle/habits such as sleep, medication, diet or physical surroundings. Furthermore, the outcomes of neural computing may also be used in building subpopulations with the same characteristics of the users', providing an opportunity to detect performance level without measuring it, being able to understand several key factors of training such as most favorable ways to peak hormones and performance in a given competition day.

In a sixth aspect, an embodiment of the present disclosure provides a data processing apparatus comprising means for carrying out the method of the first aspect or the fourth aspect.

In a seventh aspect, an embodiment of the present disclosure provides a computer-readable medium comprising instructions which, when the program is executed by a computer, cause the computer to carry out the method of the first aspect or the fourth aspect.

The present disclosure provides the aforementioned method, and the aforementioned system, for providing a recovery estimation value for user related to an exposure to a load. The disclosed method allows for the personalized estimation of load related to physical exercise, leading to a calculated recovery estimation value that is uniquely tailored to the individual user. The adaptive nature of the neural network training, coupled with the efficiency in processing user input, results in a precise and real-time assessment of the user's recovery needs. In the second aspect, the system further enhances these technical capabilities by integrating multiple neural networks within a comprehensive computing arrangement. This multi-modal approach enables the system to process diverse data types, including user responses, load measurement data, hormonal response values, and recovery estimation values. The collaboration of the first, second, and third neural networks enhances the system's capacity for holistic recovery estimation, making it a user- friendly and effective tool for personalized recovery assessments. Overall, the disclosed method and system represent a sophisticated and adaptable approach to user-centric recovery estimation, leveraging the power of neural networks for enhanced accuracy and individualization.

Throughout the present disclosure, the term “recovery estimation value” as used herein refers to a numerical value indicating the expected level of recovery needed by the user after being exposed to a load. Typical recovery estimation value is time. For example, estimation can be that a recovery time of 18 hours is needed after the exposed load (10 km running in 50 minutes for example).The recovery estimation value reflects the impact of the load on the user's physical and mental well-being and could guide recommendations for recovery strategies. In other words, the recovery estimation value represent the degree to which the user's physiological and psychological systems are affected by the load, and it may guide recommendations for recovery strategies, rest periods, or other interventions to optimize the user's well-being and performance in subsequent activities As used herein the term “load” refers to the stress or demand placed on the user, which could come from various sources such as physical exercise, mental tasks, work-related activities, or stress-inducing situations.

Optionally, the exposed load is a load related to at least one of: a physical exercise, a mental exercise, a workload, a stress load. In this regard, each load represents a distinct aspect, capturing a diverse nature of loads to which the user may be exposed. In case of physical exercise, the load may arise from activities such as running, skiing, walking, and similar endeavors. This may involve monitoring biomechanical parameters, physiological responses, and other data related to physical exertion. In some examples, sensors may be used to track heart rate, movement, or muscle activity during the physical exercise. In case of mental exercise, a load is related to cognitive or intellectual activities, including problem-solving, learning, and other mentally stimulating tasks. This may involve evaluating brain activity or cognitive performance through various means. In some other examples, cognitive tests, brain imaging, or other neuroscientific methods may be utilized to gauge mental exertion. The workload signifies the demands associated with the user's job or responsibilities, covering both physical and mental aspects. This may include assessing the complexity and demands of work-related activities. In other examples, work tasks may be analyzed using time-motion studies, workload assessments, or ergonomic evaluations. In addition, the stress load reflects the overall psychological and emotional burden on the user, arising from various sources. This may include physiological markers or psychological assessments related to stress. In some examples, heart rate variability and cortisol levels may be monitored, or psychological surveys may be used to quantify stress levels. Thus, advantageously, the method is suitable to be used for estimating the recovery estimation value for a wide range of the exposure loads.

Notably, increase in the load exposed on the user is indicative of an increase in the recovery estimation value. By using the first set of questions and the respective answers, the first neural network provides the load estimation related to the physical exercise. Further, from the load estimation, the recovery estimation value is calculated In an embodiment the recovery estimation value is calculated using intermediate step of estimating hormonal response related to the load estimation. This step improves accuracy of the recovery estimation value based on experiments.

The method further comprises providing the first set of questions to the user. Throughout the present disclosure, the term “first set of questions” refers to inquiries presented to the user to gather information about their experience with the exposed load. The questions are designed to capture relevant details that can be used to assess the impact of the load. For physical exercise, the user may be asked to rate the intensity of their recent activities, describe the type of exercise engaged in, or quantify the time dedicated to exercise per week. Mental exercise questions focus on mentally challenging tasks or projects and the cognitive demands associated with recent activities. Workload-related questions assess the overall workload, task types, and any perceived changes in workload. Stress load questions aim to gauge stress levels, identify stressors or challenges encountered, and understand the primary sources of stress, whether work-related or personal. The first set of questions is tailored to capture an understanding of the user's experiences across different load categories, forming the basis for subsequent load estimation and recovery assessment. Some examples of questions related to physical exercise that may be part of the first set of questions are “How would you rate the intensity of your recent physical activity on a scale from 1 to 10?”, “Can you describe the type of physical exercise you engaged in recently?”, “On average, how many hours per week do you dedicate to physical exercise?”.

Throughout the present disclosure, the term “computing arrangement” refers to a computational system or set of computing components that work together to perform various tasks within the described system for providing a recovery estimation value for a user. The computing arrangement encompasses the infrastructure responsible for processing data, training neural networks, and generating recovery estimations. Specifically, it includes three neural networks i.e., the first neural network, the second neural network, and the third neural network, and likely other computational elements necessary for the functioning of the system. The computing arrangement is useful in analyzing user input, physiological data, and training data to produce personalized recovery estimations.

The method further comprises receiving, after the exposure to the load, the first set of respective answers from the user, to the first set of questions. Herein, the term “first set of respective answers” refers to the responses provided by the user in response to the first set of questions. When users are presented with the first set of questions, the users are expected to answer these questions based on their own experiences and perceptions. These individual responses, specific to each question in the first set of questions, collectively form the first set of respective answers. The technical effect of receiving, after the exposure to the load, the first set of respective answers from the user is to gather specific and personalized data related to the user's experience with the load. This process enables the system to acquire detailed information about the user's response to the load, encompassing various aspects depending on the nature of the load (e.g., physical exercise, mental exercise, workload, stress load). The collected user responses serve as valuable input for subsequent analysis, particularly for the neural network models involved in load estimation and recovery assessment. In some examples, if the load pertains to physical exercise, a user might provide answers such as having engaged in a 5-kilometer run. In the case of mental exercise, responses may include working on a complex coding project. For workload-related questions, a user may express a high workload due to impending project deadlines, and for stress load, the user may rate their stress levels from 1 to 10, for example 7, citing work pressure and personal challenges.

The method further comprises using the first set of questions and the respective answers as the first input to the first neural network. The first neural network is trained to provide the load estimation (“modelled load”) related to the physical exercise using the first input. Throughout the present disclosure, the term “neural network” refers to a computational model inspired by the structure and functioning of the human brain. It is a part of the field of artificial intelligence (AI) and machine learning. Neural networks are composed of interconnected nodes, commonly referred to as neurons or artificial neurons. These networks are organized into layers, including an input layer, one or more hidden layers, and an output layer. Herein the term “first neural network” refers to a computational model that is designed to process the first set of questions and the respective answers provided by the user after the exposure to the load. It will be appreciated that the first neural network is implemented using the computing arrangement. Notably, the use of the first neural network enables to determine intricate and even non-linear relationships between the first set of questions and the respective answers to provide the load estimation. The first neural network is trained to estimate the impact of the load on the user to provide the lead estimations i.e. “modelled load”. Herein, “the training of the first neural network” involves learning the patterns and relationships between the input data (questions and answers) and the load estimation, allowing the first neural network to make predictions about the user's experience based on the provided information i.e., the first input. Herein, the term “load estimation” refers to the process of quantifying or assessing an amount of the load on the user in response to performing a physical activity. The technical effect of using the first set of question and the exposed loads is to ensure that the process is streamlined and not impact the computing arrangement involved in the process. The modelled load i.e. the load estimation refers, thus, to an estimation of actual physical load. The load estimation is based on modelling for example using a physiological model of a user in which parameters of the model are derived using the question answer pairs received from the user and load measurement data sets measured using at least one load measurement sensor. In an embodiment the load estimations are derived using a first neural network. The first neural network is trained with the load measurement data set values (such as heart beat, speed (of running for example), duration of exercises etc.) and the question answer pairs. This way correlation between the question answer pairs and actual loads provided by the load measurement data sets can be found and thus, used to provide the load estimation using the question answer pairs and the load measurement data sets as the input. Notably, the load estimation being provided using on the question answer pairs and the load measurement data sets enables to enhance an accuracy of the load estimation that is provided.

The method further comprises calculating from the load estimation the recovery estimation value. In this regard, after the first neural network has generated the load estimation using the input data provided by the user (the first set of questions and respective answers), the recovery estimation value is calculated based on this load estimation. The process involves translating the neural network's assessment of the impact of the load on the user into a quantifiable measure that represents the expected recovery needs of the user. The calculation of the recovery estimation value may involve considering various factors derived from the load estimation. Such factors may include the intensity of the load, the type of activity, and potentially other user-specific information gathered during the process. The goal is to convert the neural network's output, which represents the perceived impact of the load, into a meaningful recovery metric. For example, if the load estimation indicates a high impact from an intense physical exercise, the recovery estimation value might be higher, suggesting that the user needs more time or specific recovery strategies to recuperate adequately. As well, the dynamical role of biomarkers and hormones as a response to a given set of consecutive days' load have typically a pattern of rapid and delayed responses in values from each day, that sums up in the ways that neural computing is well suited method to recognize their combined chronological effect. Conversely, a lower load estimation might result in a lower recovery estimation value, indicating a potentially lower impact on the user, and thus, a shorter recovery period. The technical effect of the aforementioned calculation is to ensure that the information obtained from the neural network's load estimation is translated into a practical and actionable recovery estimation value, providing valuable insights for optimizing the user's recovery strategy based on their exposure to the load. In that regard the modelled load is provided from neural network which is trained to find correlation between sports computer (sensor) data and set of questions. In alternative or additional embodiment some of the questions can be related to sensor measurements as well. The questions can be arranged via application program interface (API) to aggregated sensor data after training to reinforce the first neural network training. As an example way to calculate a recovery estimation value from the load estimation: load estimation average of 100 heart beats per minute (ARH) for duration of 60 minutes (t)=100×60=600 heart beats. Then using formula RT=(AHR×t)/200. This would correspond to recovery value of 600/200 hours=30 hours.

Optionally, the method further comprises using the load estimation as a second input to a second neural network. The second neural network is trained to provide a modelled hormonal response (“modelled hormones”) related to the load estimation using the second input. Training of the second neural network comprises measuring actual loads and measuring related actual hormonal responses from a group of users and optionally from a single user to refine the model. This dataset of loads and hormonal response values are used as a training dataset. The second neural network, after training, is able to predict with high accuracy hormonal response of user by inputting a load (estimated or a measured load). This reduces amount of needed hormonal value measurements. Throughout the present disclosure, the term “second neural network” refers to another computational model, separate from the first neural network, designed to process and analyze the load estimation provided by the first neural network and then further generate a hormonal response. In this regard, the load estimation (from the first neural network) serves as the second input to the second neural network, providing information about the perceived impact of the load on the user. The second neural network is tasked with generating the modelled hormonal response based on the load estimation. Herein, the term “modelled hormonal response” refers to an artificial simulation or prediction of the hormonal changes the user may experience in response to the assessed load. In other words, the modelled hormonal responses term refers to level of hormones which are anticipated using a model. Optionally, the hormonal response comprises a cortisol value, a testosterone value and/or a ratio between the testosterone value to the cortisol value. Herein, the term “cortisol value” refers to the concentration or level of cortisol in the body at a specific point in time. The cortisol value is often measured in blood, saliva, sweat or urine to assess the body's stress response or to investigate various medical conditions. Herein, the term “testosterone value” refers to the concentration or level of testosterone in the body at a specific point in time. The testosterone value is often measured in blood tests, but can also be measured from other bodily fluids such as saliva, sweat etc. and it provides insights into the individual's hormonal balance and reproductive health. The ratio between cortisol and testosterone values provides insight into the balance between stress-related responses and anabolic (growth and repair) processes in the body. An elevated cortisol-to-testosterone ratio is often associated with increased stress or stress-related conditions. In some examples, the modelled hormones responses are taken from the experimental data. In general, hormonal response can refer to chemical changes in body.

Throughout the document, the term “hormones” is used. Classically, there's three kind of hormones: endocrinological hormones, circulating and affecting between secreting glands and target tissues, circulated via bloodstream. These hormones are the hormones that most people refer as they speak of hormones.

There's also autocrine and paracrine hormones. They affect in short/micro distances within the tissue in the extracellular fluid compartment (paracrine hormones) or even within the cell (autocrine hormones), even though they leak in measurable concentration to wider bodily tissues, too. These categories include also neurohormones, that run para- and autrcrine functions, but have also some systemic effects via the bloodstream.

In our recent embodiment, we found the use of endocrine hormones most suitable in building modeling, because anabolism and catabolismare multifactorial, massive themes taking place in the body, and they provide holistic and easy-to-collect sampling. Testosterone and cortisol act as the conductor of the orchestra, while the para-and autocrine hormones takes the place of a musician.

Furthermore, aiming to the deeper details and dynamics in the anabo-catabolic equilibrium, the use of said tissue hormones, including neurohormones as well, will provide better understanding on the sub-processes of the anabolic and catabolic state, and define for example the components of the performance and anabolism that is suffering, developing or exhausted, for example. We can define, that is it the vascular system, neural system or contractive tissue that is under highest pressure, or we can even explain that is the changes in the anabo-catabolis status due to the loss of neural drive in hypophyseal/hypothalamical areas or just the consequence for the lack of energy substrates and thus visible in insulin effector chain in the tissue hormone level. Optionally, the method further comprises the modelled hormonal response is used to refine the recovery estimation value. In this regard, the modelled hormonal response serves as additional information to better understand the physiological impact of the load on the user. By incorporating this simulated (modelled) hormonal response into the analysis, the system can potentially account for individual variations in hormonal reactions to stress or load. The technical effect of using the modelled hormonal response to refine the recovery estimation value, as described in the method, is to enhance the precision and individualization of the recovery assessment. Refined recovery estimation value thus would comprise information related to (modelled) load (via questionnaire) and (modelled) hormonal levels (modelled).

Optionally, the method further comprises providing the second set of questions to the user. Throughout the present disclosure, the term “second set of questions” refers to a supplementary series of inquiries presented to the user in the described method. These questions are designed to gather additional information beyond the first set of questions, with a focus on specific aspects related to the user's well-being. The content of these questions may vary but generally aims to provide a more detailed understanding of the user's current state, experiences, or conditions. This additional information contributes to a more comprehensive and refined recovery estimation in the context of the overall method. In an example, the second set of questions are related to sleep quality. In such example, the questions may include, but are not limited to, “how many hours of sleep did you get last night?”, and “did you experience any disruptions or difficulties in falling asleep?”. In some examples, the second set of questions are related to nutritional habits. In such example, the questions may include, but are not limited to, “describe your dietary habits today. Have you consumed a balanced diet?”, and “are there any specific foods or nutrients you've focused on for recovery?”. In some other examples, the second set of questions are related to subjective feelings. In such example, the questions may include, but are not limited to, “how do you feel overall today in terms of energy levels and mood?”, and “are there specific areas of your body or aspects of your well-being that you feel need attention?”. The technical effect of incorporating the second set of questions into the method is to enhance the depth and specificity of the information gathered from the user. This, in turn, contributes to a more detailed and personalized recovery estimation

Optionally, the method further comprises receiving the second set of respective answers from the user, to the second set of questions. Throughout the present disclosure, the term “second set of respective answers” refers to the specific responses provided by the user in relation to the additional or supplementary set of questions presented to them. In the described method, after the user is presented with the second set of questions, they are expected to provide corresponding answers to these questions. Here are examples of the second set of questions along with hypothetical responses (respective answers) related to sleep quality.

Question: “How would you rate the quality of your sleep last night on a scale from 1 to 10?”

Respective Answer: “I would rate my sleep quality as a 7.”

Optionally, the method further comprises using the second set of questions, the respective answers and the modelled hormonal response as the third input to the third neural network. The third neural network is trained to provide the modeled recovery estimation (“modelled recovery”) related to the exposed load using the third input. Throughout the present disclosure, the term “third neural network” refers to another computational model, distinct from the first and second neural networks. The third neural network takes the second set of questions, respective answers, and the modelled hormonal response as the third input for analysis. Herein, the term “modelled recovery estimation” refers to a simulated or predicted assessment of the user's recovery needs related to the exposed load, incorporating the additional information from the second set of questions and respective answers, along with the simulated hormonal response. In this regard, the third neural network is specifically trained to analyze the combined information from the second set of questions, respective answers, and the modelled hormonal response. Its purpose is to generate a modelled recovery estimation that takes into account not only the initial load estimation but also the user's responses to more detailed questions and the simulated hormonal changes. The technical effect is to refine the recovery estimation process further by incorporating a broader set of user data, including more detailed subjective information and simulated hormonal responses. The third neural network can be trained using a data set of actual hormonal values, measured loads and set of questions and answers.

Optionally, the method further comprises using the modelled recovery estimation to recalculate the refined recovery estimation to obtain the recovery estimation value to be provided to a user. Herein, the term “refined recovery estimation” refers to the recovery estimation value obtained earlier in the process, potentially based on the initial load estimation, modelled hormonal response, and any previous refinements made through the second set of questions and the third neural network. The technical effect of recalculation is to allow the recovery estimation to adapt to the most recent and comprehensive insights about the user's condition. This ensures that the provided recovery estimation value is as accurate and tailored as possible based on the evolving understanding of the user's recovery needs.

Optionally, the training of the neural network comprises generic training epochs and user dependent training epochs. Throughout the present disclosure, the term “generic training epochs” refer to the initial phase of training where the neural network learns from a diverse set of general data that is not specific to any individual user. In an implementation, the training process of the neural network involves the general training. In this regard, during the generic training epochs, the neural network is trained on a set of general data that is not specific to any individual user. This phase involves exposing the neural network to a diverse range of examples and patterns that represent a broad understanding of the task or problem it is designed to solve. The network learns general features and relationships from this diverse dataset. Throughout the present disclosure, the term “user dependent training epochs” refer to a specific phase in the training of a neural network where the model is fine-tuned and personalized based on individual user data. In another implementation, the training process of the neural network involves the user dependent training. In such implementation, the user-dependent training epochs focus on fine-tuning the neural network based on individual user data. This phase customizes the model to better understand and adapt to the specific characteristics, preferences, or patterns exhibited by each user. User-specific data, likely derived from their responses to the set of questions and other relevant information, is used to refine the neural network's parameters. The technical effect of incorporating both generic training epochs and user-dependent training epochs in the training of the neural network is to enhance the first neural network's adaptability and accuracy in providing personalized assessments.

The present disclosure also relates to the system as described above. Various embodiments and variants disclosed above, with respect to the aforementioned method, apply mutatis mutandis to the system.

Throughout the present disclosure, the term “computing arrangement” refers to a computational system or set of computing components that work together to perform various tasks within the described system for providing a recovery estimation value for a user. The computing arrangement encompasses the infrastructure responsible for processing data, training neural networks, and generating recovery estimations. Specifically, it includes three neural networks i.e., the first neural network, the second neural network, and the third neural network, and likely other computational elements necessary for the functioning of the system. The computing arrangement is useful in analyzing user input, physiological data, and training data to produce personalized recovery estimations.

Throughout the present disclosure, the term “user device” refers to a device, potentially a mobile phone, computer, or wearable, used by the user for interaction with the system. Key functions of the user device include providing the first set of questions to the user, collecting answers from the user, sending the collected answers to the computing arrangement, receiving the recovery estimation value from the computing arrangement, and presenting the recovery estimation value to the user.

Throughout the present disclosure, the term “load measurement sensor” refers to a sensor used to measure the load experienced by the user. Further, throughout the present disclosure, the term “load measurement data sets” refers to collections of data that represent measurements or readings related to the load experienced by the user, collected by the load measurement sensor. Optionally, the load measurement sensor is at least one of: a heart rate monitoring sensor, an accelerometer, and a positioning sensor. In some other implementations, the load global measurement sensor may be any other sensor or device, as per application requirements.

In some implementations, the computing arrangement includes a processor and a memory including the three neural networks. The term “processor” refers to a computational element that is operable to respond to and processes instructions that drive the system. Examples of the processor may include but are not limited to, a hardware processor, a digital signal processor (DSP), a microprocessor, and a microcontroller. The term “memory” refers to a volatile or persistent medium, such as an electrical circuit, magnetic disk, virtual memory, or optical disk, in which a computer can store data or software for any duration. Further, the user device and the computing arrangement are communicatively coupled with one another, via a communication network. Furthermore, the user device is communicatively coupled with the load measurement sensor. In some examples, the user device and the load measurement sensor may be coupled with one another, via another communication network. Herein, the term “communication network” includes a medium (e.g., a communication channel) through which the user device communicates with the computing arrangement. The communication network may be a wired or wireless communication network. Examples of the communication network may include, but are not limited to, Internet, a Local Area Network (LAN), a wireless personal area network (WPAN), a Wireless Local Area Network (WLAN), a wireless wide area network (WWAN), a cloud network, a Long-Term Evolution (LTE) network, a plain old telephone service (POTS), a Metropolitan Area Network (MAN), and/or the Internet.

Optionally, the first, the second and the third trained neural network are trained with the generic training and the user dependent training. An example neural network which have been found out to work in present dislosure is artificial neural network (ANN) that consists of 1 input layer, 3 hidden layers and 1 output layer.

As an example for the input layer is 18 node input layer. As an input for example following can be used: time, previous day hormonal values, 2 days before hormonal value and 5 heartrate zones for that day, previous day and 2 days before, related questions and answers etc. Inputs depend on which neural network (first, second or third) is considered.

Hidden layers can use Relu (Rectified Linear Unit) as the activation function.

Output layer has 1 node. The node is load estimation for the first neural network, the modelled hormonal response value for the second neural network and modelled recovery estimation for the third neural network.

In the neural network most of the calculation happens in the nodes themselves. Each node has weights for each node in the previous layer and an activation function. The node calculates a weighted sum using the values from the previous layer's nodes and the node's internal weights. This weighted sum is then fed into the activation function. In this case the used ReLu activation function only gives an output value if the input of the activation function is over 0.

Initially the neural network starts with randomized weights for each of the nodes. The network then calculates the values using weights and activation functions for each node layer by layer; while feeding the previous layer's values into the next layer. This results in an hormonal value output. In training data this output is then compared to the real value using mean square error (MSE) as loss. The loss is then backpropagated into the network by tuning he weights for each node via a gradient and the amount of tuning is dictated by the learning rate hyperparameter.

Optionally, the hormonal response values are values related to an amount of cortisol and/or testosterone.

In some embodiments, a neural network model for providing the recovery estimation value for the user and for use in the method for providing the recovery estimation value for the user related to the exposure to the load. The neural network model includes the first neural network, the second neural network, and the third neural network.

The present disclosure also relates to the method of training the neural network model as described above. Various embodiments and variants disclosed above, with respect to the aforementioned method, apply mutatis mutandis to the method of training the neural network model.

In some other embodiments, a method for training the neural network model for providing the recovery estimation value for the user is provided. The method comprises receiving a first input training dataset comprising a set of measured loads and related first sets of questions and answers and using said first input training dataset to train the first neural network. Herein, the term “first input training dataset” is a structured collection of data that includes information about measured loads and the corresponding first sets of questions and answers. The measured loads are quantifications or measurements of the physical loads experienced by users during various activities, likely related to physical exercise. Examples of the measured load may include weights lifted, distances covered, or the intensity of exercise. The first input training dataset is used to train the first neural network in the broader neural network model, enabling it to provide load estimations related to physical exercise based on user input.

The method further comprises receiving a second input training dataset comprising a set of measured loads and set of related hormonal responses and using said second input training dataset to train the second neural network. Herein, the term “second input training dataset” refers to a collection of data used to train the neural network model, specifically the second neural network in this instance. The second input training dataset includes information related to measured loads and their corresponding set of related hormonal responses. Similar to the first input training dataset, the measured loads are quantifications s or measurements of the physical loads experienced by users during various activities, likely related to physical exercise. The hormonal responses are physiological responses, specifically related to hormonal changes, that occur in the users in response to the measured loads. Examples of hormones considered may include cortisol, testosterone, or their ratio.

The method further comprises receiving a third input training dataset comprising a set of measured hormones and a second sets of question and answers and using said third input training dataset to train the third neural network. The training of the neural network model comprises generic training using data from a plurality of users to create generally trained neural networks of the neural network model and user dependent training to customize the generally trained neural networks to be user dependent. Herein, the term “third input training dataset” refers to a collection of data used to train the neural network model, specifically the third neural network. The third input training dataset includes information related to measured hormones and the second set of questions and answers. The third input training dataset is used to train the third neural network. During this training phase, the neural network learns patterns and relationships between measured hormones, the second sets of questions, and answers. The technical effect is to customize the generally trained neural networks to be more specific and personalized for individual users.

The present disclosure also relates to the data processing apparatus as described above. Various embodiments and variants disclosed above, with respect to the aforementioned method, apply mutatis mutandis to the data processing apparatus.

In some embodiments, the data processing apparatus comprising means for carrying out the method for providing the recovery estimation value for the user related to the exposure to the load and the method for training the neural network model for providing the recovery estimation value for the user. Throughout the present disclosure, the term “data processing apparatus” refers to a device or system equipped with the necessary hardware and software components to execute the method for providing recovery estimation values for a user and training the neural network model.

The present disclosure also relates to the computer-readable medium as described above. Various embodiments and variants disclosed above, with respect to the aforementioned method, apply mutatis mutandis to the computer-readable medium.

In another embodiment, a computer-readable medium comprising instructions which, when the program is executed by a computer, cause the computer to carry out the method for providing the recovery estimation value for the user related to the exposure to the load and the method for training the neural network model for providing the recovery estimation value for the user.

Optionally method comprises using provided recovery estimation value as one of the inputs to the first neural network, the second and/or the third neural network for sequential use of the method. Technical effect of this is to ensure that previous recovery values are used as input thus providing temporal aspect to coming (sequential after the previous estimation values) recovery estimation values. For example if previous estimation for recovery was 20 hours and user performs exercise during said time period that might have big impact on sequential (from the previous) recovery estimation value. Further more the system can be used to update the questions asked if the user is entering new load during recovery estimation time. The updated question could be “are you sure to do said load as you have not recovered yet”. In for this a time stamp for each asked questions and provided recovery estimations might be added. The time stamp value of previously provided recovery estimation and recovery time can be used thus to influence sequential (next round) questions. According to embodiment, when the previous recovery estimation is used as one of the inputs (to a first, second or third neural network) it is weighted based on time since the previous recovery estimation was done. As an example, if recovery time estimation was 40 hours and it was done 30 hours ago the input is weighted accordingly for example by number of (1-30/40).

One of the benefits of present disclosed system and method is that, after training, there is no need to have or carry always measurement device (such as pulse meter, GPS, sports watch) to measure load of an exercise. Answering set of questions will provide sufficient indication of recovery without measuring the load. Additionally, as the load has an impact on hormonal levels, present disclosure is beneficial as modelling correlation between load (modelled or real) and hormonal provides sufficient level of accuracy on hormonal level prediction. This way amount of needed hormonal level tests can be reduced.

### DETAILED DESCRIPTION OF THE DRAWINGS

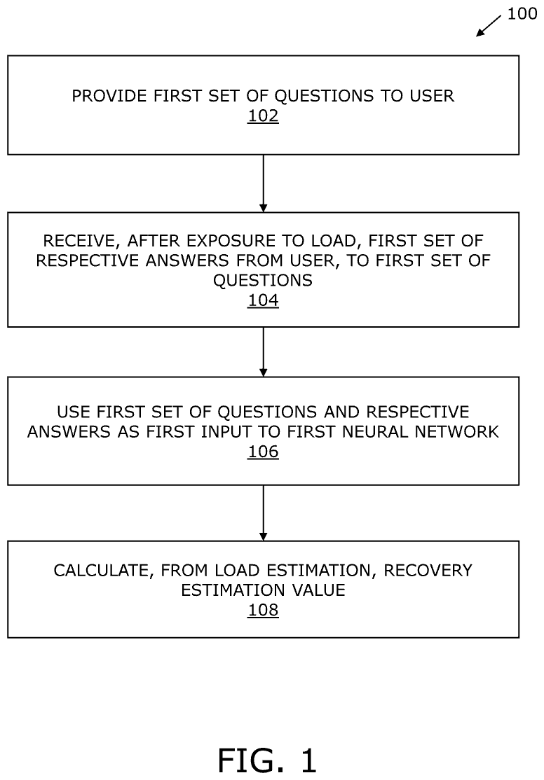

Referring to FIG. 1, illustrated is a flowchart 100 are steps of a method for providing a recovery estimation value for a user related to an exposure to a load, in accordance with an embodiment of the present disclosure. At step 102, a first set of questions is provided to the user. At step 104, a first set of respective answers to the first set of questions are received from the user after the exposure to the load. At step 106, the first set of questions and the respective answers are used as a first input to a first neural network. At step 108, a prediction request value of the at least one biomarker is measured for the first user. At step 110, the recovery estimation value is calculated from the load estimation.

The aforementioned steps 102, 104, 106, and 108 are only illustrative and other alternatives can also be provided where one or more steps are added, one or more steps are removed, or one or more steps are provided in a different sequence without departing from the scope of the claims herein.

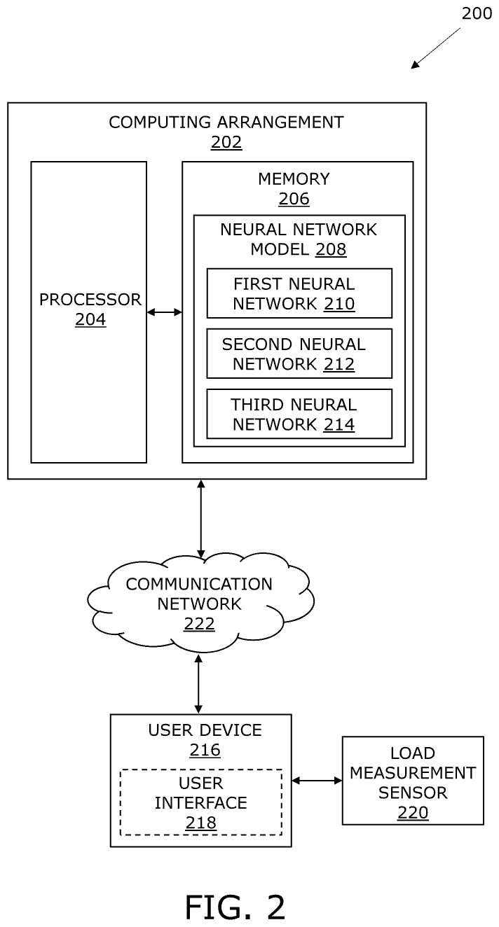

Referring to FIG. 2, illustrated is a block diagram of a system 200 for providing a recovery estimation value for a user related to an exposure to a load, in accordance with an embodiment of the present disclosure. The system 200 comprises a computing arrangement 202 comprising a processor 204 and a memory 206. In addition, the memory 206 of the computing arrangement 202 comprises a neural network model 208. The neural network model 208 comprises a first neural network 210, a second neural network 212, and a third neural network 214. The system 200 further comprises a user device 216 comprising a user interface 218. The user device 216 is communicatively coupled with a load measurement sensor 220. Moreover, the computing arrangement 202 is communicatively coupled with the user device 216, via a communication network 222.

It may be understood by a person skilled in the art that the FIG. 2 includes a simplified architecture of the system 200 for sake of clarity, which should not unduly limit the scope of the claims herein. It is to be understood that the specific implementation of the system 200 is provided as an example and is not to be construed as limiting it to specific numbers or types of servers and data repositories. The person skilled in the art will recognize many variations, alternatives, and modifications of embodiments of the present disclosure.

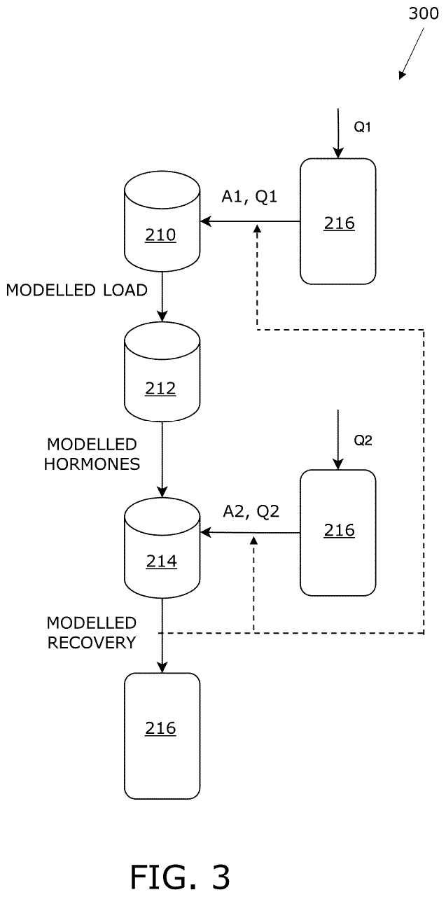

Referring to FIG. 3, illustrated is an exemplary flow diagram 300 depicting an exemplary process flow of a system for providing a recovery estimation value for a user related to an exposure to a load, in accordance with an embodiment of the present disclosure. The exemplary flow diagram 300 starts from asking a first set of questions Q1 from the user after the exposure of the load, via the user device 216 (of FIG. 2). Then, the user may provide a first set of respective answers A1 for the first set of questions Q1 through the user device 216. The first set of questions Q1 and respective answers A1 are received by the first neural network 210 as a first input. Using the first set of questions Q1 and respective answers A1, the first neural network 210 provides a modelled load. The modelled load is used to calculate a recovery estimation value. After that, the modelled load is received by the second neural network 212 as a second input. Thereafter, the second neural network 212 provides modelled hormones using the modelled load. The modelled hormones is used to refine the recovery estimation value. Then, a second set of questions Q2 is asked from the user, via the user device 216. Then, the user may provide a second set of respective answers A2 for the second set of questions Q2 through the user device 216. The second set of questions Q2 and respective answers A2 are received by the third neural network 214 as a third input. Using the modelled hormones and the second set of questions Q2 and respective answers A2, the third neural network 214 provides a modelled recovery which is later used to recalculate the recovery estimation value to a better accuracy. At last, final value of the recovery estimation value is displayed on the user device 216. Optional feedback loop is indicated with dashed line. I.e. the modelled recovery estimation (such as rest for 24 hours) can be provided as one of the input values for either to the first neural network and/or to the third neural network. The feedback loop provides thus time evolution on the modelling to take in-consideration previously modelled and proposed recovery values.

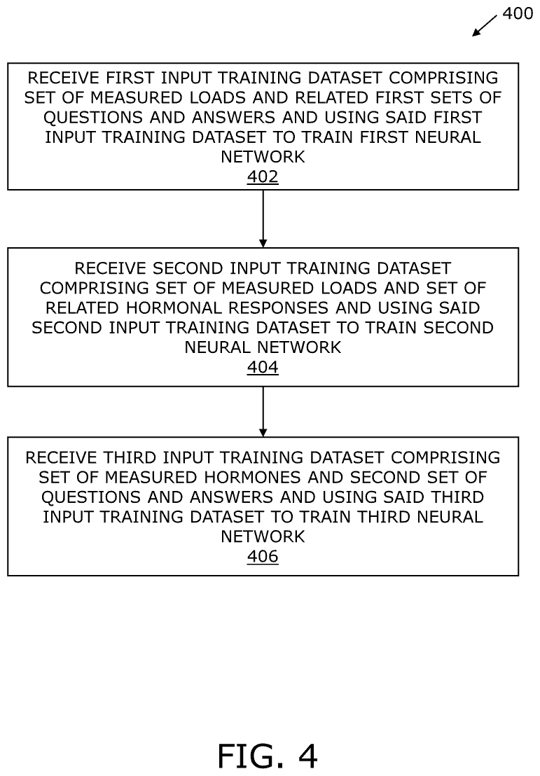

Referring to FIG. 4, illustrated is a flowchart 400 depicting steps of a method for training a neural network model for providing a recovery estimation value for a user related to an exposure to a load, in accordance with an embodiment of the present disclosure. At step 402, a first input training dataset comprising a set of measured loads and related first sets of questions and answers is received and then said first input training dataset is used to train the first neural network. At step 404, a second input training dataset comprising a set of measured loads and set of related hormonal response is received and then said second input training dataset is used to train the second neural network. At step 406 a third input training dataset comprising a set of measured hormones and a second sets of questions and answers is received and then said third input training dataset is used to train the third neural network.

The aforementioned steps 402, 404, and 406 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “includiing”, “comprising”, “incorporating”, “having” is used to describe and claim the present disclosure are intended to be construed in a non-exclusive manner, namely allowing for items, components or elements not explicitly described also to be present. Reference to the singular is also to be construed to relate to the plural.

1. A method for providing a recovery estimation value for a user related to an exposure to a load, the method comprising:
providing a first set of questions to the user, via a computing arrangement;
receiving, after the exposure to the load, a first set of respective answers from the user, to the first set of questions, at the computing arrangement;
using the first set of questions and the respective answers as a first input to a first neural network, via the computing arrangement, wherein the first neural network is trained to provide a load estimation related to the physical exercise using the first input; and
calculating from the load estimation the recovery estimation value, using the computing arrangement.

2. A method according to claim 1, wherein the method further comprises using the load estimation as a second input to a second neural network, via the computing arrangement, wherein the second neural network is trained to provide a modelled hormonal response related to the load estimation using the second input, and the modelled hormonal response is used to refine the recovery estimation value.

3. A method according to claim 2, wherein the method further comprises:
providing a second set of questions to the user, using the computing arrangement;
receiving a second set of respective answers from the user, to the second set of questions, at the computing arrangement;
using the second set of questions, the respective answers and the modelled hormonal response as a third input to a third neural network, via the computing arrangement, wherein the third neural network is trained to provide a modelled recovery estimation related to the exposed load using the third input; and
using the modelled recovery estimation to recalculate the refined recovery estimation, via the computing arrangement, to obtain the recovery estimation value to be provided to a user.

4. A method according to claim 1, wherein the exposed load is a load related to at least one of: a physical exercise, a mental exercise, a workload, a stress load.

5. A method according to claim 1, wherein the training of a neural network comprises generic training epochs and user dependent training epochs.

6. A method according to claim 2, wherein the hormonal response comprises: a cortisol value, a testosterone value and/or a ratio between the testosterone value to the cortisol value.

7. A method according to claim 2, wherein the provided recovery estimation value is used as one of the inputs to the first neural network and/or the third neural network, by the computing arrangement for sequential use of the method.

8. A system for providing a recovery estimation value for a user, the system comprising:
a computing arrangement comprising:
a first neural network, which the first neural network is trained using as training data a first set of question answer pairs, load measurement data sets from a load measurement sensor and calculated load estimations related to the first set of question answer pairs,
a second neural network, which the second neural network is trained using load estimations and related hormonal response values, and
a third neural network, which the third neural network is trained using as training data a second set of question answer pairs, hormonal response values and recovery estimation values; and
a user device for providing the first and the second set of questions to the user, collecting answers from the user, providing the collected answers to the computing arrangement, receiving from the computing arrangement the recovery estimation value and providing the recovery estimation value for the user.

9. A system according to claim 8, wherein the first, the second and the third trained neural network are trained with a generic training and a user dependent training.

10. A system according to claim 8, wherein the load measurement sensor is at least one of: a heart rate monitoring sensor, an accelerometer, a global positioning sensor.

11. A system according to claim 8, wherein the hormonal response values are values related to an amount of cortisol and/or testosterone.

12. A neural network model for providing a recovery estimation value for a user and for use in the method of claim 1, comprising:
a first neural network, wherein the first neural network is trained to provide a load estimation related to the physical exercise using a first set of questions and related answers as an input, via a computing arrangement;
a second neural network, wherein the second neural network is trained to provide a modelled hormonal response using the load estimation as an input, via the computing arrangement; and
a third neural network, wherein the third neural network is trained to provide a modelled recovery estimation related to a second set of questions and related answers, via the computing arrangement, wherein the modelled recovery estimation is used as the recovery estimation value to be provided.

13. A method for training a neural network model of claim 12 for providing a recovery estimation value for a user, wherein the method comprises:
receiving a first input training dataset comprising a set of measured loads and related first sets of questions and answers and using said first input training dataset to train the first neural network, via the computing arrangement;
receiving a second input training dataset comprising a set of measured loads and set of related hormonal responses and using said second input training dataset to train the second neural network, via the computing arrangement; and
receiving a third input training dataset comprising a set of measured hormones and a second sets of questions and answers and using said third input training dataset to train the third neural network, via the computing arrangement;
wherein,
the training of the neural network model comprises generic training using data from a plurality of users to create generally trained neural networks of the neural network model and user dependent training to customize the generally trained neural networks to be user dependent.

14. Use of the method of claim 1 for at least one of:
providing recovery estimation after physical exercise,
providing hormonal value estimations after a real or modelled load.

15. A data processing apparatus comprising means for carrying out the method of claim 1.

16. A computer-readable medium comprising instructions which, when the program is executed by a computer, cause the computer to carry out the method of claim 1.
